(19) United States
(12) Patent Application Publication
(10) Pub. No.: US 2026/0160744 A1
(43) Pub. Date: Jun. 11, 2026

(54) SYSTEMS AND METHODS FOR DETERMINING A LOCATION OF A PEST INFESTED AND/OR DISEASED PLANT

(71) Applicant: SIGNIFY HOLDING B.V., EINDHOVEN (NL)

(72) Inventors: SANGEETA MUTGI ARAVIND, MOOREBANK (AU); KAI LIU, SYDNEY (AU); CELINE CATHERINE SARAH NICOLE, EINDHOVEN (NL); MARCELLINUS PETRUS CAROLUS MICHAEL KRIJN, EINDHOVEN (NL)

(21) Appl. No.: 18/707,621

(22) PCT Filed: Nov. 7, 2022
(86) PCT No.: PCT/EP2022/080980

§ 371 Date: May 6, 2024

(30) Foreign Application Priority Data
Nov. 9, 2021 (EP) 21207074.2

(51) Int. Cl. G01N 33/00

(52) U.S. Cl. CPC G01N 33/0047; G01N 33/0098

(57) ABSTRACT

A system is disclosed for detecting a pest infestation and/or a disease on plants. The system comprises a plurality of spatially separated VOC sensors for detecting volatile organic compounds, VOCs, among a plurality of plants. The system also comprises a database system having stored thereon a plurality of indications of respective distress VOCs and/or response VOCs. Herein, each distress VOC is a VOC that is emitted by a pest infested and/or diseased plant and each response VOC is a VOC that is emitted by a plant in response to the plant detecting a distress VOC from a pest infested and/or diseased plant. The system also comprises a data processing system that is configured to-receive from the plurality of sensors one or more signals indicative of a detected VOC and indicative of at least one location where the detected VOC is detected, and to-based on the one or more signals from the plurality of VOC sensors, determine that the detected VOC corresponds to a distress VOC stored in the database system or determine that the detected VOC corresponds to a response VOC stored in the database system, and to-based on determining that a distress VOC or response VOC has been detected, and based on the at least one location as indicated in the one or more signals, determine a location of a pest infested and/or diseased plant.

### FIELD OF THE INVENTION

This disclosure relates to a system and a method determining a location of a pest infested and/or diseased plant. In particular to such a system and method wherein distress and/or response VOCs are detected by a plurality of spatially separated VOC sensors.

### BACKGROUND

Research has uncovered that plants release Volatile Organic Compounds (VOCs) when in duress either due to a pest infestation or due to a disease, e.g., due to an infection by pathogens. Plants have evolved to release these unique compounds as a defensive mechanism to attract other carnivorous insects to control pests and as a form of plant-to-plant communication. See for example {Exploiting Plant Volatile Organic Compounds (VOCs) in Agriculture to Improve Sustainable Defense Strategies and Productivity of Crops by Brilli et al, Front. Plant Sci., 19 Mar. 2019} and {Vivaldo, G., Masi, E., Taiti, C. et al. The network of plants volatile organic compounds. Sci Rep 7, 11050 (2017). https://doi.org/10.1038/s41598-017-10975-x}.

It has also been suggested that VOCs can be used for early detection of insect infestation and pathogen infection. See for example {A CMUT-Based Electronic Nose for Real-Time Monitoring of Volatiles Emitted by Plants: Preliminary Results by Mahmud et al., 2018 IEEE SENSORS, DOI: 10.1109/ICSENS.2018.8589740}.

US 2019/0170718 A1 discloses a multi-sensor device comprising a housing containing multiple sensor modules for capturing and transmitting sensor data for plants in a crop. A control unit within the housing is operable to control the sensor modules, and a communications interface is connected to the control unit for transmitting data from said plurality of sensor modules. The sensor modules can include a physiological sensor, a surface analysis sensor, and chemical sensor. The multi-sensor device can be used as a hand-held device or mounted to a mobile platform for use in an automated crop monitoring system.

However, there is a need in the art for more improved systems and methods for detecting a pest infestation and/or a disease on plants.

### SUMMARY

To that end, a system is disclosed for detecting a pest infestation and/or a disease on plants. The system comprises a plurality of spatially separated VOC sensors for detecting volatile organic compounds, VOCs, among a plurality of plants. The plurality of spatially separated VOC sensors are preferably arranged to be spatially distributed amongst the plurality of plants, thereby covering a sensing area substantially corresponding to the area of the plurality of plants. That is, the plurality of spatially separated VOC sensors are adapted to be spatially distributed, e.g., in a grid, across a sensing area substantially corresponding to an area comprising by the plurality of plants The system also comprises a database system having stored thereon a plurality of indications of respective distress VOCs and/or response VOCs. Herein, each distress VOC is a VOC that is emitted by a pest infested and/or diseased plant and each response VOC is a VOC that is emitted by a plant in response to the plant detecting a distress VOC from a pest infested and/or diseased plant. The system also comprises a data processing system that is configured to
receive from the plurality of sensors one or more signals indicative of a detected VOC and indicative of at least one location where the detected VOC is detected, and to
based on the one or more signals from the plurality of VOC sensors, determine that the detected VOC corresponds to a distress VOC stored in the database system or determine that the detected VOC corresponds to a response VOC stored in the database system, and to
based on determining that a distress VOC or response VOC has been detected, and based on the at least one location as indicated in the one or more signals, determine a location of a pest infested and/or diseased plant.

Due to the plurality of VOC sensors, the system enables to localize plants that are (likely) diseased and/or pest infested. This is highly beneficial as it allows to take counter measures against the pest infestation and/or disease at specific locations. Plants that have been diseased may for example be removed from the plurality of plants so that they will not infect other plants.

The localization of the diseased and/or pest infested plant(s) based on location information of detected VOCs may be implemented in various ways. For example, if the detected VOCs are distress VOCs, the location of the respective VOC sensors provides an indication of diseased and/or pest infested plant(s). In addition, a respective magnitude of the detected distress VOCs, as may be included in the respective sensor signals, may allow to localize the source/nest/hearth of the disease and/or pest infestation, e.g., by using the principles of (tri)lateration or fingerprinting. If the detected VOCs are response VOCs, the locations of the VOC sensors detecting the response VOCs are indicative of locations surrounding a diseased and/or pest infested plant(s). In such case, the location of the diseased and/or pest infested plant(s) can be determined by using the principle of the centroid or geometric center for all the locations of the respective VOC sensors detecting the response VOCs. Such methods may also benefit from respective magnitudes of detected response VOCs, for example by determining a center of gravity (instead of a geometric center) defined as the weighted mean of the sensor locations weighted by the respective magnitude of the detected response VOC at the sensor location. It will be clear to the skilled reader that, the more locations are available where response VOCs are detected, the more accurate the location of diseased and/or pest infested plant(s) can be determined. The skilled person will understand that any other suitable location determination method may equally be used.

Each VOC sensor may output a signal that also comprises an indication of its location. This indication may be embodied as an identifier of the particular VOC sensor. The data processing system may have stored the location of the VOC sensor in the plant growing facility, e.g., a greenhouse, which then allows to determine the location where the VOC is detected based on the identifier of the VOC. Hence, the one or more signals from the plurality of sensors, being indicative of at least one location where the detected VOC is detected, may be implemented as the VOC sensors including their respective identifiers in the one or more signals that they provide to the data processing system.

Distress VOCs may refer to VOCs the emission of which is caused by a plant being diseased and/or pest infested. A response VOC may be understood as a VOC that is emitted by a plant in response to the plant detecting such distress VOC.

Detecting a distress VOC may be performed by detecting a quantity of a VOC higher or lower than a threshold quantity. To illustrate, it may be that a plant emits a particular VOC “A” at a first rate if it is in a healthy state, however, when it is diseased and/or pest infested, it emits VOC “A” at a second rate higher or lower than the first rate. Thus, based on the detected quantity of VOC “A” being higher or lower than some appropriately selected threshold value, it may be determined that the detected VOC “A” is a distress VOC since its emission is at least partially caused by the plant in question being diseased and/or pest infested.

It could also be that some VOC, e.g., VOC “B”, is only emitted by a plant if it is diseased and/or pest infested. In such case, detecting the distress VOC “B” may be simply performed by detecting the presence of VOC “B”.

Likewise, detecting a response VOC may be performed by detecting a quantity of the VOC higher or lower than a threshold quantity and/or by simply detecting the presence of the VOC.

The plurality of VOC sensors may be spatially distributed among the plurality of plants, preferably substantially evenly distributed.

The data processing system may be configured to output a signal indicative of the determined location of the pest infested and/or diseased plant. This output signal may be output to other modules of the system, e.g., as a control signal which causes these other modules to perform one or more steps. As will be discussed further below, such control signal may be used to control a disease mitigation system and/or to a pest infestation mitigation system. Alternatively or additionally, the signal is embodied as a perceivable warning signal configured to notify a human user of the location of a pest infested and/or diseased plant. Such warning signal may be a visual signal and/or audio signal.

The VOC sensors may be any sensor known in the art that can detect at least one VOC. It should be appreciated that a VOC sensor may output a very complex signal from which detected VOCs cannot be readily derived. Such complex signal may comprise a fingerprint of detected VOCs in the sense that a computer-implemented method is performed for determining, based on the signal as output by the VOC sensor, which one or more VOCs have been detected. The computer-implemented method for determining which one or more VOCs have been detected, optionally which quantity of such VOCs has been detected, may be based on a model obtained using machine learning algorithms. The model may be used to link the signal as output by a VOC sensor to one or more VOCs and optionally to their respective quantities. The machine learning algorithm may build the model based on training data. Herein, the training data may comprise a plurality of, preferably many, reference signals as output by VOC sensors. The training data then preferably indicates for each reference signal one or more associated VOCs and preferably their respective quantities. Any machine learning algorithm can then be used to build a suitable model for determining one or more detected VOCs based on signals as output by a VOC sensor.

Once a detected VOC has been identified, the data processing system can search in the database system whether the identified VOC is labeled as a distress VOC and/or response VOC.

In an embodiment, the database system has stored thereon a plurality of indications of respective response VOCs. In such embodiment, the data processing system is configured to, based on the one or more signals from the plurality of VOC sensors, determine that the detected VOC corresponds a response VOC stored in the database system, and to, based on determining that a response VOC has been detected, and based on the at least one location as indicated in the one or more signals, determine a location of a pest infested and/or diseased plant.

Healthy, unstressed plants can emit so-called response VOCs in response to detecting VOCs that are emitted by a neighbouring pest infested and/or diseased plant. See for example {Falik O, Mordoch Y, Quansah L, Fait A, Novoplansky A (2011) Rumor Has It . . . : Relay Communication of Stress Cues in Plants. PLOS ONE 6(11): e23625. https://doi.org/10.1371/journal.pone.0023625}. The inventors have found that diseases and pest infestations may be more easily detected based on detecting response VOCs than directly detecting distress VOCs that are emitted by diseased and/or pest infested plants. Plants that are diseased and/or pest infested may namely be too weak to emit (large quantities of) distress VOCs, which may render the detection of such distress VOCs difficult. Further, especially at the beginning of a disease and/or pest infestation, when only few plants have been infected, there are likely more healthy plants emitting response VOCs than there are plants emitting distress VOCs. To illustrate, if only one plant is pest infested, then only this plant will emit distress VOCs. However, this single plant typically has several healthy neighboring plants that may all emit response VOCs. For this reason too, there may be higher quantities of response VOCs than distress VOCs as a result of which response VOCs can be more easily detected. Through this mechanism, pest infestations and/or diseases can be detected early in a reliable manner.

In an embodiment, the one or more signals of the detected VOC are indicative of a plurality of locations where the VOC is detected. In such embodiment, the data processing system may be configured to, based on the plurality of locations indicated in the one or more signals, determine one or more locations where pest infested and/or diseased plants are present.

The use of a plurality of spatially separated VOC sensors wherein the plurality of spatially separated VOC sensors are spatially distributed across a sensing area substantially corresponding to an area comprising the plurality of plants and wherein location information of the detected VOCs is available allows to generate a heat map, which indicates in which regions plants are pest infested and/or diseased. Such heat map provides a real-time overview with real-time sensor data, e.g., to the grower, of the health status of the plurality of plants.

Of course, more than one VOC may be detected, and for each detected VOC one or more locations may be indicated where the VOC in question is measured.

In an embodiment, at least one VOC sensor of the plurality of VOC sensors is configured to detect VOCs and/or substances of VOCs using gas-chromatography and/or using mass-spectroscopy and/or using gas chromatography-mass spectrometry and/or using proton transfer reaction time-of-flight mass spectrometry. At least one VOC sensor of the plurality of VOC sensors may comprise an electronic nose and/or a smartphone-based VOC sensor.

An electronic nose is an electronic sensing device intended to detect odors or flavors. Essentially such instrument consists of head space sampling, a chemical sensor array, and pattern recognition modules, to generate signal patterns that are used for characterizing odors. Electronic noses include three major parts: a sample delivery system, a detection system, a computing system. The sample delivery system enables the generation of the headspace (volatile compounds) of a sample, which is the fraction analyzed. The system then injects this headspace into the detection system of the electronic nose. The sample delivery system is essential to guarantee constant operating conditions. The detection system, which consists of a sensor set, is the “reactive” part of the instrument. When in contact with volatile compounds, the sensors react, which means they experience a change of electrical properties. In most electronic noses, each sensor is sensitive to all volatile molecules but each in their specific way. However, in bio-electronic noses, receptor proteins which respond to specific odor molecules are used. Most electronic noses use chemical sensor arrays that react to volatile compounds on contact: the adsorption of volatile compounds on the sensor surface causes a physical change of the sensor. A specific response is recorded by the electronic interface transforming the signal into a digital value. Recorded data are then computed based on statistical models. An electronic nose may include a metal-oxide semiconductor (MOSFET) device, which may be understood to be a transistor used for amplifying or switching electronic signals. This works on the principle that molecules entering the sensor area will be charged either positively or negatively, which should have a direct effect on the electric field inside the MOSFET. Thus, introducing each additional charged particle will directly affect the transistor in a unique way, producing a change in the MOSFET signal that can then be interpreted by pattern recognition computer systems. So essentially each detectable molecule will have its own unique signal for a computer system to interpret. Additionally or alternatively, the electronic nose may include conducting polymers. Additionally or alternatively, the electronic nose may include polymer composites, which may be understood similar in use to conducting polymers but formulated of non-conducting polymers with the addition of conducting material such as carbon black. Additionally or alternatively, the electronic nose may include quartz crystal microbalance (QCM), which may be understood to be configured to measure mass per unit area by measuring the change in frequency of a quartz crystal resonator. This can be stored in a database and used for future reference. Additionally or alternatively, the electronic nose may include surface acoustic wave (SAW), which may be understood a class of microelectromechanical systems (MEMS) which rely on the modulation of surface acoustic waves to sense a physical phenomenon. Additionally or alternatively, the electronic nose may include, optionally miniaturized, surface mass spectrometers.

A VOC sensors may be a VOC sensor as described in R. N. Pudake et al. (eds.), Biosensors in Agriculture: Recent Trends and Future Perspectives, Concepts and Strategies in Plant Sciences, https://doi.org/10.1007/978-3-030-66165-6_5, especially the VOC sensors described in chapter 5 of this reference.

In an embodiment, the system comprises one or more humidity sensors for measuring a humidity of air. In such embodiment, the data processing system may be configured to receive from the one or more humidity sensors one or more signals indicative of a measured humidity. The data processing system may further be configured to, based on the one or more signals from the one or more humidity sensors, and based on the determined location of the pest infested and/or diseased plant, predict for a future time instance at which one or more locations one or more plants of the plurality of plants will be/become diseased and/or pest infested.

Preferably, the one or more signals indicative of measured humidity also indicate a location where the humidity was measured. The prediction for the future time instance at which one or more locations one or more plants of the plurality of plants will be/become diseased and/or pest infested may be performed further based on the location where the humidity was measured. The one or more signals may be indicative of respective humidity values for respective locations. In such case, the prediction for the future time instance at which one or more locations one or more plants of the plurality of plants will be/become diseased and/or pest infested may be performed further based on the indicated respective locations and associated humidity values.

This embodiment enables to accurately predict how the disease and/or pest will spread. To illustrate, some pests can develop faster if the humidity is at certain levels. Being able to accurately predict the spread of the disease/pest, allows to take anticipatory countermeasures to mitigate the spread of the disease/pest infestation.

In an embodiment, the system comprises one or more temperature sensors for measuring a temperature. In such embodiment, the data processing system may be configured to receive from the one or more temperature sensors one or more signals indicative of a measured temperature. The data processing system may further be configured to, based on the one or more signals from the one or more temperature sensors, and based on the determined location of the pest infested and/or diseased plant, predict, for a future time instance, at which one or more locations one or more plants of the plurality of plants will be/become diseased and/or pest infested.

Preferably, the one or more signals indicative of measured temperature also indicate a location where the temperature was measured. The prediction for the future time instance at which one or more locations one or more plants of the plurality of plants will be/become diseased and/or pest infested may be performed further based on the location where the temperature was measured. The one or more signals may be indicative of respective temperature values for respective locations. In such case, the prediction for the future time instance at which one or more locations one or more plants of the plurality of plants will be/become diseased and/or pest infested may be performed further based on the indicated respective locations and associated temperature values.

The inventors have found that temperature is also an important parameter for predicting the spread of a disease and/or pest. Hence, this embodiment enables to accurately predict the spread.

In an embodiment, the system comprises one or more air flow sensors for measuring direction of air flow. In such embodiment, the data processing system may be configured to receive from the one or more air flow sensors one or more signals indicative of a direction of air flow. The data processing system may further be configured to, based on the one or more signals from the one or more air flow sensors, and based on the determined location of the pest infested and/or diseased plant, predict, for a future time instance, at which one or more locations one or more plants of the plurality of plants will be/become diseased and/or pest infested.

Preferably, the one or more signals indicative of measured air flow also indicate a location where the air flow was measured. Also, preferably, these signals indicate both a direction and magnitude of air flow. The prediction for the future time instance at which one or more locations one or more plants of the plurality of plants will be/become diseased and/or pest infested may be performed further based on the location where the air flow was measured. The one or more signals may be indicative of respective air flow values for respective locations. In such case, the prediction for the future time instance at which one or more locations one or more plants of the plurality of plants will be/become diseased and/or pest infested may be performed further based on the indicated respective locations and associated airflow values. Each airflow value may be indicative of both a direction and magnitude of air flow.

These embodiments enable to accurately predict at which locations plants are likely going to suffer from disease or pest infestation. The spread of a diseases and/or pest may namely be influenced by the air flow among the plurality of plants. Typically, if at a location of a diseased and/or pest infested plant, the air flows substantially in a particular direction, then likely the diseased and/or pest will spread faster in that particular direction.

Embodiments in which the data processing system can predict for a future time at which one or more locations one or more plants of the plurality of plants will be/become diseased and/or pest infested enable to take effective measures against the pest and/or disease and the pest and/or disease spread. For example, plants which are (likely) going to be infected may be protected, e.g., by moving them or by applying other counter measures. Of course, the data processing system may be configured to predict this for a plurality of future times, so that a development of the pest and/or disease over a certain future time period may be predicted.

The data processing system may predict which one or more plants will be/become diseased and/or pest infested by predicting in which regions, e.g. in a greenhouse, the pest and/or disease will have evolved at the future time.

In an embodiment, the data processing system is configured to, based on the determined location of the pest infested and/or diseased plant, determine a location where measures are to be effected for mitigating the pest infestation and/or disease, and/or to determine one or more measures for mitigating the pest infestation and/or disease.

In an embodiment, the data processing system is configured to, based on the one or more signals indicative of a detected VOC, determine which disease and/or pest infestation is present among the plurality of plants, for example based on which specific VOC(s) are detected by the plurality of VOC sensors. It may be that certain diseases/pest infestations cause specific distress VOC(s) and/or specific response (VOC(s) to be emitted. Of course, which disease and/or pest infestation is present among the plurality of plants may also be determined by a user visiting the determined location of a pest infested and/or diseased plant in order to inspect, e.g., visually inspect, the plants at that location in order to determine the type of disease/pest infestation. In any case, the data processing system may be configured to determine one or more countermeasures based on a determined type of disease and/or pest infestation.

Depending on the mitigation measures, they may be effected at the location of the diseased/pest infested plant and/or at another location. To illustrate, plants surrounding the diseased and/or pest infested plant may be removed so that the disease/pest cannot transfer further to other plants.

In an embodiment, the data processing system is configured to send a control signal to a disease mitigation system and/or to a pest infestation mitigation system. The control signal may cause these systems to mitigate the disease infection and/or pest infestation.

This embodiment advantageously enables to perform mitigating actions for inhibiting the spreading of the disease and/or pest infestation.

The data processing system may be configured to determine a risk for the plurality of plants of becoming diseased and/or becoming infected by a pest. In such case, the data processing system may be configured to compare the risk to a threshold value and send the control signal based on a determination that the determined risk is higher than said threshold value.

In an embodiment, the data processing system is configured to, based on the one or more locations at which one or more plants are predicted to be/become diseased and/or pest infested for the future time instance, determine a location where measures are to be effected for mitigating the pest infestation and/or disease.

This embodiment enables to take anticipatory countermeasures. If for example a disease is predicted to spread very fast into some particular region of the greenhouse, then additional countermeasures are preferably taken in this particular region.

In an embodiment, the control signal indicates the location where measures are to be effected. The disease mitigation system and/or to a pest infestation mitigation system may then be configured to take countermeasures specifically at the indicated position, e.g. in order to prevent a further outbreak.

In an embodiment, the system for determining a location of a pest infested and/or diseased plant further comprises the system for mitigating a pest infestation and/or a disease in a plurality of plants. The system for mitigating may further comprise at least one of
a pesticide provisioning system configured to provide pesticide, preferably to provide pesticide at selected positions,
a fungicide provisioning system configured to provide fungicide, preferably to provide fungicide at selected positions,
a disinfection radiation system configured to provide disinfection radiation, such as UV C light, preferably to provide disinfection radiation at selected positions,
a humidity adjustment system for influencing humidity, preferably at selected position, and
a temperature control system for influencing temperature, preferably at selected positions.

In such embodiment, the data processing system may be configured to send a control signal to respectively the pesticide provisioning system and/or fungicide provisioning system and/or disinfection radiation system and/or humidity adjustment system and/or temperature control system, the control signal causing these systems to mitigate the disease infection and/or pest infestation, or the spread thereof.

One aspect of this disclosure relates to a computer-implemented method for use in a system for determining a location of a pest infested and/or diseased plant, the method comprising
receiving, from a plurality of spatially separated VOC sensors for detecting volatile organic compounds, VOCs, among a plurality of plants, the plurality of spatially separated VOC sensors spatially distributed across a sensing area substantially corresponding to an area comprising the plurality of plants, one or more signals indicative of a detected VOC and indicative of at least one location where the detected VOC is detected, and to
based on the one or more signals from the plurality of VOC sensors, determining that the detected VOC corresponds to a distress VOC as stored in a database system or determine that the detected VOC corresponds to a response VOC as stored in the database system, the database system having stored thereon a plurality of indications of respective distress VOCs and/or response VOCs, wherein each distress VOC is a VOC that is emitted by a pest infested and/or diseased plant and each response VOC is a VOC that is emitted by a plant in response to the plant detecting a distress VOC from a pest infested and/or diseased plant, and to -based on determining that a distress VOC or response VOC has been detected, and based on the at least one location as indicated in the one or more signals, determining a location of a pest infested and/or diseased plant.

One aspect of this disclosure relates to a data processing apparatus comprising means for carrying out any of the methods described herein for detecting a pest infestation and/or disease on plants described herein.

One aspect of this disclosure relates to a computer program comprising instructions which, when the program is executed by a computer, cause the computer to carry out any of the methods described herein for detecting a pest infestation and/or disease on plants described herein.

One aspect of this disclosure relates to a non-transitory computer-readable medium having stored thereon any of the computer programs described herein.

One aspect of this disclosure relates to a computer comprising
a computer readable storage medium having computer readable program code embodied therewith, and
a processor, preferably a microprocessor, coupled to the computer readable storage medium, wherein responsive to executing the computer readable program code, the processor is configured to carry out any of the methods described herein for detecting a pest infestation and/or disease on plants described herein.

One aspect of this disclosure relates to a computer program or suite of computer programs comprising at least one software code portion or a computer program product storing at least one software code portion, the software code portion, when run on a computer system, being configured for executing out any of the methods described herein for detecting a pest infestation and/or disease on plants described herein.

One aspect of this disclosure relates to a non-transitory computer-readable storage medium storing at least one software code portion, the software code portion, when executed or processed by a computer, is configured to perform out any of the methods described herein for detecting a pest infestation and/or disease on plants described herein.

As will be appreciated by one skilled in the art, aspects of the present invention may be embodied as a system, a method or a computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Moreover, a computer program for carrying out the methods described herein, as well as a non-transitory computer readable storage-medium storing the computer program are provided. A computer program may, for example, be downloaded (updated) to the existing data processing systems or be stored upon manufacturing of these systems.

Elements and aspects discussed for or in relation with a particular embodiment may be suitably combined with elements and aspects of other embodiments, unless explicitly stated otherwise. Embodiments of the present invention will be further illustrated with reference to the attached drawings, which schematically will show embodiments according to the invention. It will be understood that the present invention is not in any way restricted to these specific embodiments.

### BRIEF DESCRIPTION OF THE DRAWINGS

Aspects of the invention will be explained in greater detail by reference to exemplary embodiments shown in the drawings, in which:

FIG. 1A illustrates a system for detecting a pest infestation and/or a disease on plants according to an embodiment;

FIG. 1B illustrates data stored in a database system according to an embodiment;

FIG. 2 illustrates how a VOC may be detected using machine learning techniques according to embodiment;

FIGS. 3-5 illustrates data stored in a database system according to an embodiment;

FIG. 6 illustrates an embodiment of the system that comprises a plurality of environmental sensors;

FIG. 7 depicts a heat map indicating diseased and/or pest infested plants for a time t=1 according to an embodiment;

FIG. 8 depicts a heat map indicating diseased and/or pest infested plants for a time t=2 according to an embodiment;

FIG. 9 an embodiment of the system wherein the data processing system is configured to send a control signal to a disease mitigation system and/or to a pest infestation mitigation system;

FIG. 10 illustrates a data processing system according to an embodiment.

### DETAILED DESCRIPTION OF THE DRAWINGS

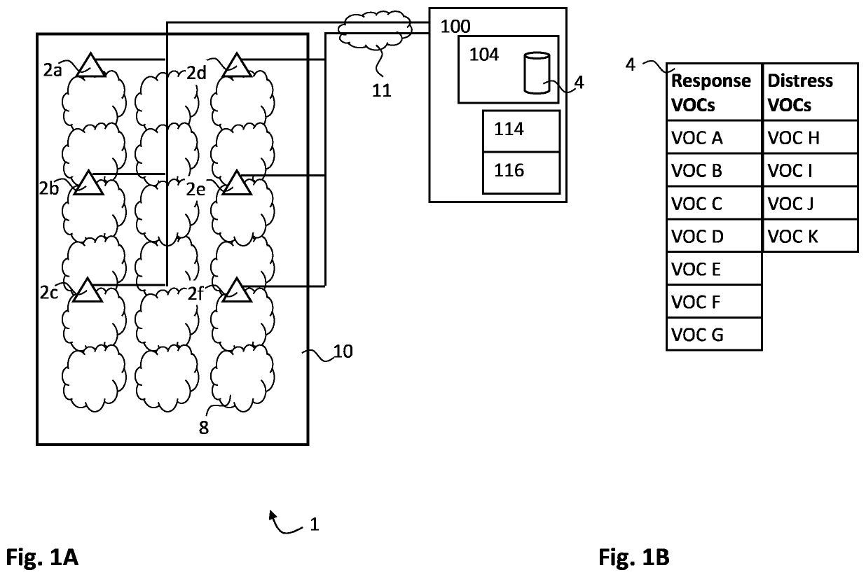

In the figure identical reference number indicate identical or similar elements. FIG. 1A illustrates a system 1 for determining a location of a pest infested and/or diseased plant among a plurality of plants 8, according to an embodiment. The system may be implemented in a greenhouse 10 in order to monitor the health of the plants 8 grown in the greenhouse. However, it should be appreciated that the system may also be implemented in an outdoor environment or fully closed indoor environment as long as the distress VOCs and/or response VOCs can be detected. The greenhouse 10 may comprise means for controlling the environmental parameters inside the greenhouse, such as temperature, humidity, air flow, et cetera. Such means may be used to create optimal conditions for the plants 8 to grow. However, such means may also function as a pest and/or disease mitigation system as described herein.

The system 1 comprises a plurality of spatially separated VOC sensors 2 for detecting volatile organic compounds, VOCs, among a plurality of plants 8. FIG. 1 shows six sensors 2a . . . 2f, however, any number of sensors may be used. In principle, the more sensors are used, the more accurate, in terms of location, the diseased and/or pest infested plants can be detected. Using more VOC sensors in principle increases the spatial resolution with which diseased and/or pest infested plants can be detected. A VOC sensor as used herein may be any device that can measure a VOC in the sense that upon a particular VOC interacting with the VOC sensor, the VOC sensor can output a signal based on which the detected VOC can be identified. Such VOC sensor may be adapted to respond only to a particular VOC or may be adapted to respond to a plurality of different VOCs. A VOC sensor may be use technology based on gas-chromatography and/or mass-spectroscopy and/or gas chromatography-mass spectrometry.

The system 1 also comprises a database system 4 having stored thereon a plurality of indications of respective distress and/or response VOCs. Each distress VOC is a VOC that is emitted by a pest infested and/or diseased plant. Each response VOC is a VOC that is emitted by a plant in response to the plant detecting a distress VOC from a pest infested and/or diseased plant. FIG. 1B illustrates the concept. The database system comprises several indications of VOCs, in FIG. 1B “VOC A”, “VOC B”, et cetera. Such VOC indications may have been obtained by previous study of distress/response VOCs of the plants in question and/or may be known from literature and may be used to identify the detected VOC. In FIG. 1, the database system 4 is part of a data processing system 100, however, the database system 4 may also be separate from the data processing system 100.

The data processing system 100 is configured to receive from the plurality of sensors 2 one or more signals indicative of a detected VOC. These signals are also indicative of at least one location where the detected VOC is detected. The data processing system may receive these signals from the sensors via or wireless and/or wired connection. The solid lines in FIG. 1A between the data processing system 100 and the VOC sensors 2 indicate such connections. The data processing system 100 is also configured to, based on the one or more signals from the plurality of VOC sensors, determine that the detected VOC corresponds to a distress and/or response VOC stored in the database system. For this, the data processing system 100 for example performs a lookup function in the database 4 in order to check whether the detected VOC is listed in the database 4.

The data processing system 100 is configured to, based on the determination that the detected VOC corresponds to a distress or response VOC stored in database system 4 and based on the at least one location as indicated in the one or more signals received from the plurality of sensors, determine a location of a pest infested and/or diseased plant.

The data processing system may output a signal indicative of the determined location. Such signal may be any kind of signal. In an example, the data processing system comprises output device 114, for example a display and/or a loudspeaker, that can output a signal. Additionally or alternatively, the data processing system 100 comprises a network device 116 via which the data processing system 100 can communicate with other devices.

The data processing system 100 may output the signal to other devices, for example to a pest and/or disease mitigation system, that can take mitigating measures based on the signal from the data processing system. The data processing system 100 may be configured to, based on the determined location of the pest infested and/or diseased plant, determine a location where measures are to be effected for mitigating the pest infestation and/or disease and/or determine one or more measures for mitigating the pest infestation and/or disease.

The data processing system 100 is shown in FIG. 1A as a separate system. However, it should be appreciated that the data processing system 100 may be a distributed system in the sense that elements of the data processing system may be remote from each other. For example, it could also be that each VOC sensor comprises a copy of the database 4 and an own processor that checks whether the detected VOC is a distress and/or response VOC. In such case, the data processing system 100 is thus a distributed system with functionality that is distributed among the plurality of VOC sensors.

Optionally, the data processing system sits remote from the VOC sensors 2 in the sense that the VOC sensors transmit their signals to the data processing system 100 via a network such as the internet 11. In such case, the data processing system 100 may be understood to be implemented as a remote server.

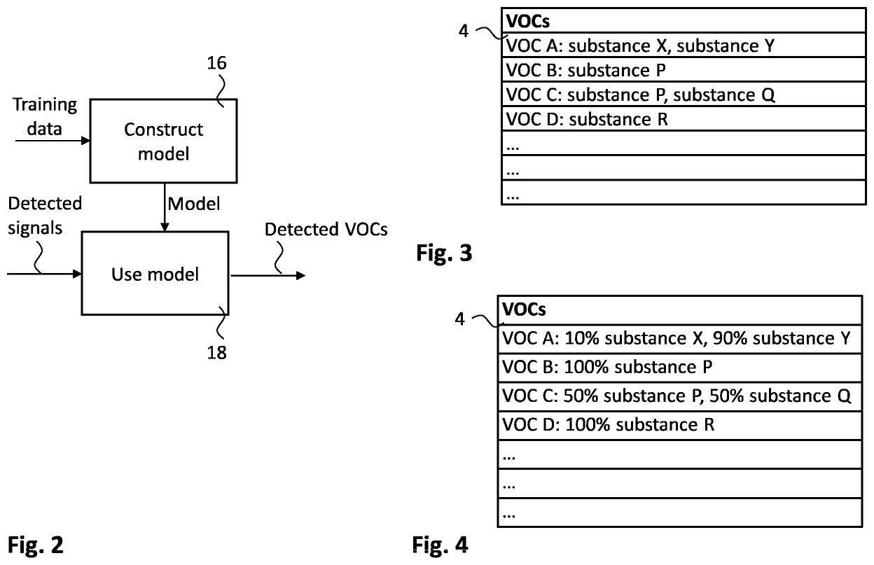

FIG. 2 illustrates how a VOC may be detected using machine learning techniques. In a first step 16, a model is constructed based on training data as shown. The training data indicate, for each of a plurality of signals from arbitrary VOC sensors, not necessarily the plurality of VOC sensors that are eventually used, the one or more VOCs that were actually present at and detected by the VOC sensor in question. By performing machine learning techniques known in the art, a model can be constructed that links incoming signals from VOC sensors, which signals may be quite complex, to one or more VOCs.

Once the model has been constructed, it can be used, see step 18, to determine which one or more VOCs are present at a particular VOC sensor based on signals output by the particular VOC sensor, i.e., to identify the one or more VOCs that are present at the VOC sensor.

For the systems and methods disclosed herein, it is less relevant how a VOC is detected. It is important, though, that detected VOCs are identified so that it can be checked whether they are distress and/or response VOCs.

FIG. 3 illustrates a database system 4 that may be used in an embodiment. In this embodiment, each indication of a VOC as stored in the database system 4 indicates one or more substances comprised in the detected VOC in question. To illustrate, VOC A consists of substance X and a substance Y, whereas VOC B only consists of substance P. In such embodiment, preferably, the plurality of VOC sensors is configured to detect substances of volatile organic compounds and the one or more signals from the plurality of VOC sensors indicate one or more detected substances comprised in the detected VOC. This allows the data processing system 100 to match the one or more detected substances as indicated by the one or more signals to one or more substances as indicated by the indication for the VOC stored in the database system. This may improve the accuracy with which VOCs may be identified. However, the embodiment described with reference to FIG. 3 may also cause some difficulty for the data processing system 100 to distinguish VOC B from VOC C for example, because these VOCs both comprise substance P.

In order to even further improve the detection capabilities of the system, the plurality of VOC sensors may be configured to detect quantities of substances of volatile organic compounds as well. The one or more signals from the plurality of VOC sensors may indicate one or more detected quantities of respective one or more substances comprised in the detected VOC. Then, the database system 4 of FIG. 4 may be used, in which each indication of a VOC indicates one or more quantities of respective one or more substances comprised in the VOC in question. Then, the data processing system 100 can match the one or more detected quantities of respective one or more substances as indicated by the one or more signals to one or more quantities of respective one or more substances as indicated by the indication for the VOC stored in the database system 4 in order to determine which VOC if present at and detected by the sensor. In this manner, the data processing system will for example be able distinguish VOC B from VOC C.

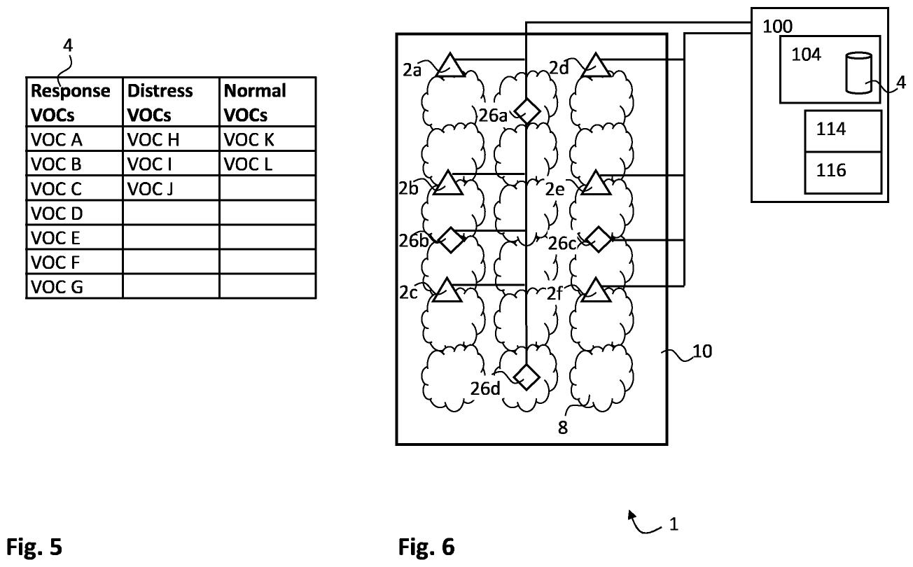

FIG. 5 illustrates that the database system may have also stored thereon a plurality of indications of respective normal VOCs. This allows the data processing system 100 to detect normal VOCs as well. It should be appreciated that the normal VOCs can be detected in the same manner as the response VOCs can be detected. Normal VOCs may be understood as VOC that a healthy plant emits when surrounded by other healthy plants, i.e., without the plant responding to a detected distress VOC from another plant.

FIG. 6 illustrates an embodiment of the system 1 that comprises a plurality of environmental sensors 26. FIG. 6 shows four environmental sensors 26a . . . 26d, however, any number of environmental sensors may be used. The plurality of environmental sensors may comprise one or more humidity sensors for measuring a humidity of air and/or one or more temperature sensors for measuring a temperature of air and/or on or more air flow sensors for measuring direction of air flow. The system may thus comprise humidity sensors as well as temperature sensors as well as air flow sensors. The data processing system 100 is configured to receive from the plurality of environmental sensors one or more signals indicative of a measured humidity and/or indicative of a measured temperature and/or indicative of a direction of air flow. These signals may be received in the same way as how the signals output by the VOC sensors are received, i.e., via a wired or wireless connection.

As said, the one or more signals from the plurality of VOC sensors may be indicative of a position at which the detected VOC was detected, for example by the signals identifying from which sensor the signal originates, wherein the data processing system 100 has a database having stored thereon the different VOC sensors and their positions. In a similar way, the one or more signals from the plurality of environmental sensor 26 may be indicative of a position at which the environmental sensor is located, for example by the signals identifying from which sensor the signal originates, wherein the data processing system 100 has a database having stored thereon the different environmental sensors and their positions.

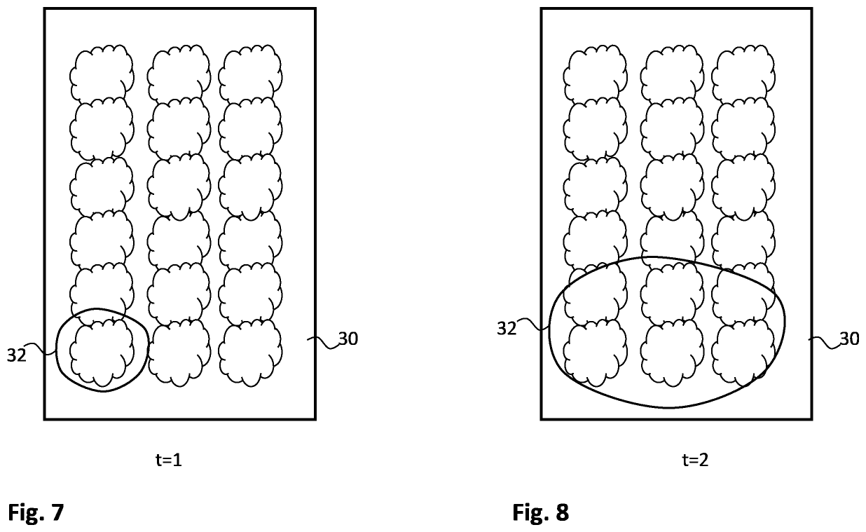

This is advantageous in that the location of pest infested and/or diseased plants can be determined and for example allows to construct a heat map 30 as depicted in FIGS. 7 and 8 that illustrates at which locations plants are diseased and/or pest infested. In addition, the location of the environmental sensors allows to construct a heat map for the humidity, temperature and/or air flow in the greenhouse, such heat maps enabling a better understanding how (direction) and how fast (time) detected diseases and/or pest could spread in the greenhouse, in turn enabling a better disease and/or pest mitigation control.

In FIG. 7, the heat map indicates an area 32 in which the plants are diseased and/or pest infested for a time t=1, which may be a current time. Thus, at time t=1, only the plants in the bottom left corner of the greenhouse are diseased and/or pest infested. This is valuable information because it allows to take countermeasures against the pest and/or disease specifically in area 32.

In an embodiment, data processing system is configured to, based on the indicated position of the detected VOC, and preferably also based on one or more signals from one or more environmental sensors described herein, predict for a future time at which one or more location one or more plants out of the plurality of plants will be/may become diseased and/or pest infested. In particular, the data processing system 100 may be configured to construct a heat map 30 for a future time t=2 as depicted in FIG. 8. The example shown in FIG. 8 illustrates that it is predicted that at time t=2, the plants in area 32 are/may be pest infested and/or diseased. It should be appreciated that this prediction may be performed under the assumption that no countermeasures are taken.

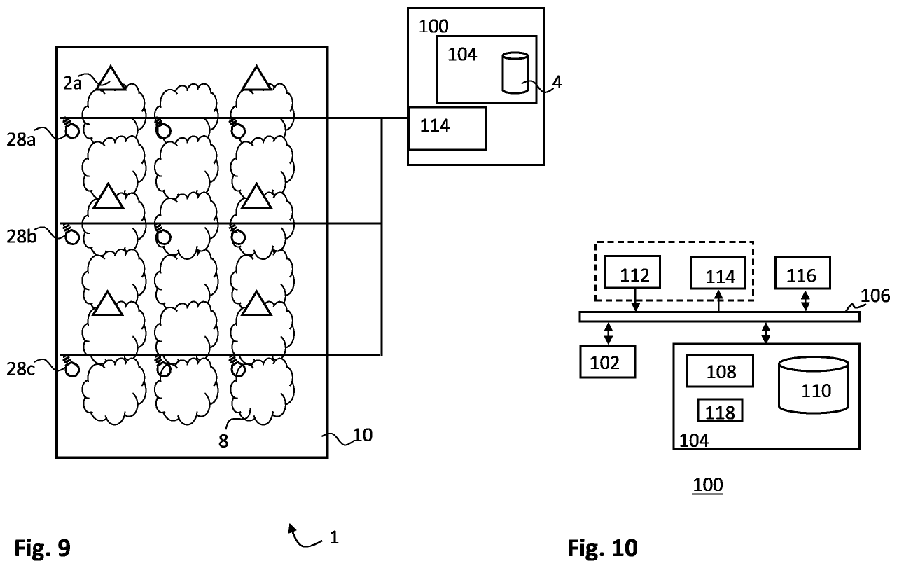

FIG. 9 illustrates an embodiment of the system 1 wherein the data processing system 100 is configured to send a control signal to a disease mitigation system and/or to a pest infestation mitigation system 28, e.g., 28a . . . 28c, the control signal causing these systems 28 to mitigate the disease infection and/or pest infestation.

The disease mitigation system and/or pest mitigation system 28 may comprise any one or more of
a pesticide provisioning system configured to provide pesticide, preferably to provided pesticide at selected positions, and/or
a fungicide provisioning system configured to provide fungicide, preferably to provided fungicide at selected positions, and/or
a disinfection radiation system configured to provide disinfection radiation, such as UVC light, preferably to provided disinfection radiation at selected positions, and/or
a humidity adjustment system for influencing humidity, preferably at selected position, and/or
a temperature control system for influencing temperature, preferably at selected positions.

Preferably, the data processing system indicates the position near and/or at which the response VOC has been detected and/or indicates one or more positions of the respective one or more plants that have been predicted to be/become diseased and/or pest infested at the future time. This allows the mitigation system 28 to perform mitigating actions specifically at these locations.

FIG. 10 depicts a block diagram illustrating a data processing system according to an embodiment.

As shown in FIG. 10, the data processing system 100 may include at least one processor 102 coupled to memory elements 104 through a system bus 106. As such, the data processing system may store program code within memory elements 104. Further, the processor 102 may execute the program code accessed from the memory elements 104 via a system bus 106. In one aspect, the data processing system may be implemented as a computer that is suitable for storing and/or executing program code. It should be appreciated, however, that the data processing system 100 may be implemented in the form of any system including a processor and a memory that is capable of performing the functions described within this specification.

The memory elements 104 may include one or more physical memory devices such as, for example, local memory 108 and one or more bulk storage devices 110. The local memory may refer to random access memory or other non-persistent memory device(s) generally used during actual execution of the program code. A bulk storage device may be implemented as a hard drive or other persistent data storage device. The processing system 100 may also include one or more cache memories (not shown) that provide temporary storage of at least some program code in order to reduce the number of times program code must be retrieved from the bulk storage device 110 during execution.

Input/output (I/O) devices depicted as an input device 112 and an output device 114 optionally can be coupled to the data processing system. Examples of input devices may include, but are not limited to, a keyboard, a pointing device such as a mouse, a touch-sensitive display, one or more VOC sensors 2 as described herein, one or more environmental sensors 26 as described herein, or the like. Examples of output devices may include, but are not limited to, a monitor or a display, speakers, pest mitigation systems 28 as described herein, or the like. Input and/or output devices may be coupled to the data processing system either directly or through intervening I/O controllers.

In an embodiment, the input and the output devices may be implemented as a combined input/output device (illustrated in FIG. 10 with a dashed line surrounding the input device 112 and the output device 114). An example of such a combined device is a touch sensitive display, also sometimes referred to as a “touch screen display” or simply “touch screen”. In such an embodiment, input to the device may be provided by a movement of a physical object, such as, e.g., a stylus or a finger of a user, on or near the touch screen display.

A network adapter 116 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 100, and a data transmitter for transmitting data from the data processing system 100 to said systems, devices and/or networks. Modems, cable modems, and Ethernet cards are examples of different types of network adapter that may be used with the data processing system 100.

As pictured in FIG. 10, the memory elements 104 may store an application 118. In various embodiments, the application 118 may be stored in the local memory 108, the one or more bulk storage devices 110, or apart from the local memory and the bulk storage devices. It should be appreciated that the data processing system 100 may further execute an operating system (not shown in FIG. 10) that can facilitate execution of the application 118.

The application 118, being implemented in the form of executable program code, can be executed by the data processing system 100, e.g., by the processor 102. Responsive to executing the application, the data processing system 100 may be configured to perform one or more operations or method steps described herein.

In another aspect, the data processing system 100 may represent a client data processing system. In that case, the application 118 may represent a client application that, when executed, configures the data processing system 100 to perform the various functions described herein with reference to a “client”. Examples of a client can include, but are not limited to, a personal computer, a portable computer, a mobile phone, or the like.

In yet another aspect, the data processing system 100 may represent a server. For example, the data processing system may represent an (HTTP) server, in which case the application 118, when executed, may configure the data processing system to perform (HTTP) server operations.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 102 described herein.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of embodiments of the present invention has been presented for purposes of illustration but is not intended to be exhaustive or limited to the implementations in the form disclosed. Many modifications and variations will be apparent to those of ordinary skill in the art. The embodiments were chosen and described in order to best explain the principles and some practical applications of the present invention, and to enable others of ordinary skill in the art to understand the present invention for various embodiments with various modifications as are suited to the particular use contemplated.

1. A system for determining a location of a pest infested and/or diseased plant, the system comprising:
a plurality of spatially separated VOC sensors for detecting volatile organic compounds, VOCs, among a plurality of plants, the plurality of spatially separated VOC sensors adapted to be spatially distributed across a sensing area substantially corresponding to an area comprising the plurality of plants, and
a database system having stored thereon a plurality of indications of respective distress VOCs and/or response VOCs, wherein each distress VOC is a VOC that is emitted by a pest infested and/or diseased plant and each response VOC is a VOC that is emitted by a plant in response to the plant detecting a distress VOC from a pest infested and/or diseased plant, and
a data processing system that is configured to:
receive from the plurality of sensors one or more signals indicative of a detected VOC and indicative of at least one location where the detected VOC is detected, and to
based on the one or more signals from the plurality of VOC sensors, determine that the detected VOC corresponds to a distress VOC stored in the database system or determine that the detected VOC corresponds to a response VOC stored in the database system, and to
based on determining that a distress VOC or response VOC has been detected, and based on the at least one location as indicated in the one or more signals, determine a location of a pest infested and/or diseased plant.

2. The system according to claim 1, wherein the database system has stored thereon a plurality of indications of respective response VOCs, and wherein
the data processing system is configured to, based on the one or more signals from the plurality of VOC sensors, determine that the detected VOC corresponds to a response VOC stored in the database system, and to
based on determining that a response VOC has been detected, and based on the at least one location as indicated in the one or more signals, determine a location of a pest infested and/or diseased plant.

3. The system according to claim 1, wherein the one or more signals of the detected VOC are indicative of one or more locations where the VOC is detected, wherein the data processing system is configured to:
based on the one or more locations indicated in the one or more signals, determine one or more locations where pest infested and/or diseased plants are present.

4. The system according to claim 1, wherein at least one VOC sensor of the plurality of VOC sensors is configured to detect VOCs using gas-chromatography and/or using mass-spectroscopy and/or using gas chromatography-mass spectrometry and/or using proton transfer reaction time-of-flight mass spectrometry and/or comprises an electronic nose.

5. The system according to claim 1, further comprising one or more humidity sensors for measuring a humidity of air, wherein
the data processing system is configured to receive from the one or more humidity sensors one or more signals indicative of a measured humidity, wherein
the data processing system is configured to, based on the one or more signals from the one or more humidity sensors, and based on the determined location of the pest infested and/or diseased plant, predict, for a future time instance, at which one or more locations one or more plants of the plurality of plants will be diseased and/or pest infested.

6. The system according to claim 1, further comprising one or more temperature sensors for measuring an air temperature, wherein
the data processing system is configured to receive from the one or more temperature sensors one or more signals indicative of a measured temperature, wherein
the data processing system is configured to, based on the one or more signals from the one or more temperature sensors, and based on the determined location of the pest infested and/or diseased plant, predict, for a future time instance, at which one or more locations one or more plants of the plurality of plants will be diseased and/or pest infested.

7. The system according to claim 1, further comprising one or more air flow sensors for measuring direction of air flow, wherein
the data processing system is configured to receive from the one or more air flow sensors one or more signals indicative of a direction of air flow, wherein
the data processing system is configured to, based on the one or more signals from the one or more air flow sensors, and based on the determined location of the pest infested and/or diseased plant, predict, for a future time instance, at which one or more locations one or more plants of the plurality of plants will be diseased and/or pest infested.

8. The system according to claim 1, wherein the data processing system is configured to, based on the determined location of the pest infested and/or diseased plant, determine one or more measures for mitigating the pest infestation and/or disease.

9. The system according to claim 8, wherein the data processing system is configured to send a control signal to a disease mitigation system and/or to a pest infestation mitigation system, the control signal causing these systems to mitigate the disease infection and/or pest infestation.

10. The system according to claim 8, wherein the data processing system is configured to, based on the one or more locations at which one or more plants are predicted to be diseased and/or pest infested for the future time instance, determine a location where measures are to be effected for mitigating the pest infestation and/or disease.

11. The system according to claim 10, wherein the control signal indicates the location where measures are to be effected.

12. The system according to claim 9, further comprising the system for mitigating a pest infestation and/or a disease in a plurality of plants, the system for mitigating further comprising at least one of:
a pesticide provisioning system configured to provide pesticide, preferably to provide pesticide at selected positions,
a fungicide provisioning system configured to provide fungicide, preferably to provide fungicide at selected positions,
a disinfection radiation system configured to provide disinfection radiation, such as UV-C light, preferably to provide disinfection radiation at selected positions,
a humidity adjustment system for influencing humidity, preferably at selected position, and
a temperature control system for influencing temperature, preferably at selected positions, wherein the data processing system is configured to send the control signal to the respective at least one of pesticide provisioning system, fungicide provisioning system, disinfection radiation system, humidity adjustment system and temperature control system, the control signal causing these respective systems to mitigate the disease infection and/or pest infestation.

13. A computer-implemented method for use in a system for determining a location of a pest infested and/or diseased plant, the method comprising:
receiving, from a plurality of spatially separated VOC sensors for detecting volatile organic compounds, VOCs, among a plurality of plants, the plurality of spatially separated VOC sensors spatially distributed across a sensing area substantially corresponding to an area comprising the plurality of plants, one or more signals indicative of a detected VOC and indicative of at least one location where the detected VOC is detected, and to
based on the one or more signals from the plurality of VOC sensors, determining that the detected VOC corresponds to a distress VOC stored in a database system or determine that the detected VOC corresponds to a response VOC stored in the database system, the database system having stored thereon a plurality of indications of respective distress VOCs and/or response VOCs, wherein each distress VOC is a VOC that is emitted by a pest infested and/or diseased plant and each response VOC is a VOC that is emitted by a plant in response to the plant detecting a distress VOC from a pest infested and/or diseased plant, and to
based on determining that a distress VOC or response VOC has been detected, and based on the at least one location as indicated in the one or more signals, determining a location of a pest infested and/or diseased plant.

14. (canceled)

15. A non-transitory computer readable medium comprising instructions which, when the instructions are executed by a data processing system, causes the data processing system to perform the method of claim 13.
